(19) United States
(12) Patent Application Publication
(10) Pub. No.: US 2007/0112018 A1
(43) Pub. Date: May 17, 2007

(54) Treatment of peripheral arterial occlusive disease

(76) Inventor: Andrew Sternlicht, Chestnut Hill, MA (US)

Correspondence Address: CLARK & ELBING LLP, 101 FEDERAL STREET, BOSTON, MA, 02110, US

(21) Appl. No.: 11/543,595

(22) Filed: Oct. 5, 2006

Related U.S. Application Data
(60) Provisional application No. 60/724,857, filed on Oct. 6, 2005.
(60) Provisional application No. 60/735,969, filed on Nov. 10, 2005.

(51) Int. Cl. A61K 31/4745

(52) U.S. Cl. 514/291

(57) ABSTRACT

The invention relates to the treatment of peripherial arterial occlusive disease.

### CROSS-REFERENCE TO RELATED APPLICATIONS

This application claims benefit from U.S. Provisional Application No. 60/724,857, filed Oct. 6, 2005, and U.S. Provisional Application No. 60/735,969, filed Nov. 10, 2005, each of which is hereby incorporated by reference.

### BACKGROUND OF THE INVENTION

Atherosclerosis and its complications lead to half of all adult deaths in the United States and other western societies, and its incidence is increasing in developing countries. Evidence suggesting that atherosclerosis is a chronic inflammatory disease has led to considerable research into the role played by infectious agents. Although a range of viruses and bacteria have been implicated in atherosclerosis, Chlamydia (C.) pneumoniae shows the strongest association to date in a range of epidemiological and experiment-based studies.

Peripheral arterial occlusive disease (PAOD; also referred to as peripheral arterial disease (PAD)) results either from atherosclerotic or inflammatory processes producing arterial stenosis, or from thrombus formation associated with underlying atherosclerotic disease. A common site for PAOD is in the lower limbs. This process of atherosclerosis causes intimal thickening and plaque formation encroaching the arterial lumen, decreasing the effective luminal radius of afflicted arterial segments, producing an anatomic and sometimes functional obstruction to blood flow. When these conditions arise, an increase in vascular resistance can lead to a reduction in distal perfusion pressure and blood flow. PAOD affects 20% to 30% of men and women age 50 years and older seen in general medical practices, and is associated with other forms of coronary artery disease, specifically atherosclerosis and general functional impairments (e.g., slower walking ability or decreased endurance) and may have a significant negative impact on the quality of independent living. PAOD can be reliably detected with doppler-recorded systolic pressures as a differential in the ankle-brachial ratio of these pressures.

Research by others revealed a marked risk of cardiovascular morbidity and mortality in the 5 years after diagnosis of intermittent claudication, the primary symptom of PAOD. The American Heart Association and the National Cholesterol Education Program recommend intensive intervention because of the increased risk of cardiovascular events associated with PAOD. The use of cholesterol-lowering drugs, antiplatelet therapy, thienopyridine (e.g., ticlopidine and clopidogrel) drugs, angiotensin converting enzyme inhibitors, beta blockers, pharmacologic treatment of intermittent claudicating symptoms (pentoxifylline, cilostazol, and estrogen replacement therapy), and exercise have all been explored. However, some of these interventions involve risk, especially in subpopulations of PAOD patients, and in some cases, provide no clinical benefit.

Numerous recent studies suggest a possible role of C. pneumoniae in PAOD. C. pneumoniae is an obligate intracellular prokaryotic pathogen and is a common causative pathogen of many acute upper and lower respiratory tract infections, which are often self-limiting and subclinical. Unlike the other major human chlamydial pathogen, C. trachomatis, C. pneumoniae can infect and survive in a wider range of host cell types, such as lung epithelium, resident macrophages, circulating monocytes, arterial smooth muscle cells, and vascular endothelium. Since exposure to C. pneumoniae is extremely common, infections occur repeatedly throughout life for most people. Treatment of chronic Chlamydia infections can be difficult as the life cycle of the organism includes resident time in morphologic forms not susceptible to antibiotics.

Clinical studies have been reported which explored the possible role of C. pneumoniae in PAOD and the role that its eradication may play in patient outcome. Others have demonstrated the presence of C. pneumoniae and seropositivity in atheromatous plaques in patients with PAOD. Krayenbuehl and colleagues studied the effectiveness of roxithromycin, an antibiotic with both anti-chlamydial and some anti-inflammatory properties, in treating C. pneumoniae associated with PAOD in a 4-year prospective study investigating clinical outcome parameters. The study confirmed that patients who were C. pneumoniae seropositive had a worse clinical course of PAOD than patients who were seronegative.

In the original publication of this data by Wiesli and colleagues, a statistically significant benefit for daily use of roxithromycin for 28 days in prevention of progression of lower limb atherosclerosis for almost 3 years was seen in C. pneumoniae seropositive men. Treatment with roxithromycin also reduced the number of invasive revascularization procedures. Additionally, over a 6-month period, this treatment resulted in a statistically significant reduction of soft plaque components in carotid arteries. High-titered C. pneumoniae seropositivity was significantly associated with clinical progression of lower limb atherosclerosis and the need for invasive revascularization during the 2.7-year observation period. Importantly, results of the Wiesli study also indicated that the beneficial effects of roxithromycin could be ascribed to antimicrobial, rather than anti-inflammatory, properties of the drug.

Vainas and colleagues conducted a randomized study of short-term (i.e. 3 days) use of a low dose of azithromycin (300 mg) versus placebo in more than 500 patients with PAOD. The results, gathered after 2 years of follow-up, showed that a short-term course of antibiotics offered no significant reduction of morbidity or mortality in PAOD patients. However, seropositivity to chlamydia at baseline entry into the study was associated with a significantly increased risk of developing either a combined endpoint including PAOD related complications or an exclusively PAOD-related event over the two year follow up period (p=0.02 and 0.01 respectively.) Interestingly, chlamydia seropositivity at baseline was not significantly associated with the probability of reaching a cardiac event (p=0.69) during the follow-up period. The researchers admitted that the lack of beneficial effect may have been related to inadequate medication (too low a dose for too short a time period) and/or to suboptimal patient selection (lack of seropositivity to chlamydia inclusion criteria). Differences in the beneficial patient outcomes in the Vainas study versus those of Wiesli, were further attributed by Wiesli to the percentage of patients with severe impairment of peripheral arterial circulation at baseline entry (20% in the Vainas study, and 65% in the Wiesli study).

### SUMMARY OF THE INVENTION

In general, the present invention is based on our discovery that treatment with rifalazil resulted in reduced C. pneumoniae burden and plaque area stenosis in an animal rabbit model of atherosclerosis in which C. pneumoniae infection exacerbated plaque deposition, compared with placebo-treated animals. Based on this observation, rifalazil and other rifamycins are therefore useful for the treatment of PAOD. Accordingly, the invention features a method of treating PAOD in a patient in need thereof (i.e., a patient diagnosed as having PAOD or at risk for developing PAOD) by administering to the patient a rifamycin in an amount effective to treat PAOD in the patient. In one embodiment, the patient has not been diagnosed as having a bacterial infection that can be treated by administration of a rifamycin. In another embodiment, the patient has been diagnosed as having an infection of C. pneumoniae. In another embodiment, the patient is seropositive for C. pneumoniae (i.e., having an IgG antibody titers ≧1:64, on an microimmunofluorescence assay). A patient is considered to be treated if any one of the following conditions achieves significant improvement: (1) ankle brachial index (ABI) at baseline either compared to the patient prior to treatment or to the aggregate performance of patients treated with placebo; (2) peak walking time (PWT) at baseline compared with that measured at time of assessment either compared to themselves or the aggregate performance of patients treated with placebo; (3) fuictional measures of performance (e.g., the SF-36 or Walking Impairment Questionnaire) at baseline either compared to the patient prior to treatment or to the aggregate performance of patients treated with placebo.

The invention also features a method of increasing the peak walking time (PWT) (defined as the maximum time in minutes and seconds walked on a treadmill until severe claudication symptoms forces the cessation of exercise) in a patient in need thereof by administering to the patient a rifamycin in an amount effective to increase the PWT. In one embodiment, the patient has not been diagnosed as having a bacterial infection that can be treated by administration of a rifamycin. In another embodiment, the patient has been diagnosed as having an infection of C. pneumoniae. In another embodiment, the patient is seropositive for C. pneumoniae.

The invention also features a method for increasing the painless walking distance (PWD) in a patient in need thereof by administering to the patient a rifamycin in an amount effective to increase the PWD. In one embodiment, the patient has not been diagnosed as having a bacterial infection that can be treated by administration of a rifamycin. In another embodiment, the patient has been diagnosed as having an infection of C. pneumoniae. In another embodiment, the patient is seropositive for C. pneumoniae.

The invention also features method for:

(i) reducing the occurrence and/or severity of intermittent claudication;

(ii) reducing the functional impairments associated with the progression of PAOD;

(iii) reducing the number and/or frequency of vascular interventions over time and related clinical complications over time;

(iv) reducing the number and/or frequency of cardiovascular complications over time;

(v) reducing localized inflammation in an atherosclerotic plaque;

(vi) reducing the size of an atherosclerotic plaque;

(vii) reducing the level of one or more inflammatory biomarkers (e.g., C-reactive protein, IL-6, IL-11, lipoprotein-associated phospholipase A2, fractalkine, monocyte chemotactic protein 1, neopterin, tumor necrosis factor receptors I and II, selectin, fibrinogen, ICAM-1, VCAM-1, myeloperoxidase);

(viii) reducing the clinical complications associated with angioplasty and/or stent placement;

(ix) reducing intimal hyperplasia and in-stent and peri-stent restenosis that occur after stent placement;

(x) reducing vascular smooth muscle cell proliferation and/or the cellular and molecular products of vascular smooth muscle cell proliferation (including those mediated by the Toll-Like Receptor-2 pathways; and

(xi) restoring endothelial function and capability in a patient. Each of these methods involves administering an effective amount of a rifamycin (i.e., an amount sufficient to achieve the desired result).

In one embodiment of any of these methods, the patient has not been diagnosed as having a bacterial infection that can be treated by administration of a rifamycin. In another embodiment, the patient has been diagnosed as having an infection of C. pneumoniae. In another embodiment, the patient is seropositive for C. pneumoniae.

In any of the foregoing aspects, a preferred rifamycin is rifalazil. The dosage of rifalazil normally ranges between 0.001 mg to 100 mg, preferably is 1-50 mg, or more preferably 2-25 mg. The rifalazil may be given daily (e.g., a single oral dose of 0.001 mg to 100 mg/day, preferably 2.5 to 25 mg/day) or less frequently (e.g., a single oral dose of 5 mg/week, 12.5 mg/week, or 25 mg/week). Treatment may be given for a period of one day to one year, or longer. In one embodiment, the rifalazil is administered once per week in an amount of between 12.5 and 25 mg/week for 4-10 weeks (e.g., 8 weeks). This protocol may be repeated periodically (e.g., every 3, 6, or 12 months) for up to the lifetime of the patient. In another embodiment, a rifamycin is administered at an initial dose of 2.5 mg to 100 mg for one to seven consecutive days, followed by a maintenance dose of 0.005 mg to 10 mg once every one to seven days for one month, one year, or even for the life of the patient. In another embodiment, a rifamycin is administered at an initial dose of 2.5 to 100 mg once a week, for a period of two to 16 weeks, followed by a dose of 2.5 to 50 mg once a week, once each two weeks, once a month, or once each two months, for a period of months to years, or even for the remaining lifespan of a patient.

The rifamycin can be a rifamycin other than rifalazil. The dosage of rifampin, rifabutin, rifapentin, or rifaximin normally ranges between 50 to 1000 mg/day. These rifamycins may be given daily (e.g., a single oral dose of 50 to 600 mg/day) or less frequently (e.g., a single oral dose of 50, 100, or 300 mg/week). Treatment may be administered for a period of one day to one year, or even longer. In one embodiment, one of these rifamycins is administered at an initial dose of 600 mg to 2000 mg for one to seven consecutive days, followed by a maintenance dose of 100 mg to 600 mg once every one to seven days for one month, one year, or even for the life of the patient.

If desired, a rifamycin may be administered in conjunction with one or more additional agents such as anti-inflammatory agents, e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) steroids (e.g., cortisone, dexamethasone, hydrocortisone, methylprednisolone, prednisolone, prednisone, triamcinolone), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These secondary therapeutic agents may be administered within 14 days, 7 days, 1 day, 12 hours, or 1 hour of administration of a rifamycin, or simultaneously therewith. The additional therapeutic agents may be present in the same or different pharmaceutical compositions as the rifamycin of the invention. When present in different pharmaceutical compositions, different routes of administration may be used. For example, rifalazil may be administered orally, while a second agent may be administered by intravenous, intramuscular, or subcutaneous injection.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

The “peak walking time” or “PWT” is defined as the maximum time in minutes and seconds walked on a treadmill until severe claudication symptoms forces the cessation of exercise. The treadmill test is conducted at a constant speed of 2 mph with a 2% increase in grade every 2 minutes. The treadmill test begins at 2 mph, 0% grade and subsequent increases in grade must be made with a programmable treadmill up to a maximum grade of 18%. Patients are made familiar with the treadmill before the test.

“Painless walking distance” or “PWD” means the maximum distance walked on a treadmill until severe claudication symptoms forces the cessation of exercise. The treadmill test is conducted at a constant speed, with the treadmill grade fixed at a horizontal (flat) level.

The “claudication onset time” or “COT” is defined as the time in minutes and seconds walked on a treadmill until the onset of claudication symptoms regardless of whether this is manifested as muscle pain, ache, cramps, numbness, or faigue. This does not include joint pain or other pain not associated with claudication.

An “Exercise Treadmill Test” or “ETT” utilizing the Gardner protocol (Table 1) is used to assess the patient's COT and PWT. The COT for both lower extremities is recorded. The PWT and the particular lower extremity causing the subject to stop the ETT are recorded.

TABLE 1

| | Speed | Elevation | Time |
|---|---|---|---|
| Stage | (mph) | (% grade) | (min) |
| Rest | 2.0 | 0 | — |
| 1 | 2.0 | 0 | 2 minutes |
| 2 | 2.0 | 2 | 2 minutes |
| 3 | 2.0 | 4 | 2 minutes |
| 4 | 2.0 | 6 | 2 minutes |
| 5 | 2.0 | 8 | 2 minutes |
| 6 | 2.0 | 10 | 2 minutes |
| 7 | 2.0 | 12 | 2 minutes |
| 8 | 2.0 | 14 | 2 minutes |
| 9 | 2.0 | 16 | 2 minutes |
| 10 | 2.0 | 18 | 2 minutes |
| 11 | 2.0 | 18 | At least 20 minutes |
| Recovery | 0.0 | 0 | N/A |

The “ankle-brachial index” or “ABI” is defmed as the ratio between the higher of the two pedal systolic blood pressure (dorsalis pedis and posterior tibial) and the higher of the two systolic brachial pressures. A continuous wave Doppler, between 5 and 10 MHz, is used to measure the systolic pressures in both the dorsalis pedis and posterior tibial arteries in each leg, as well as the brachial arteries in each arm. The higher of the two arm pressures and the higher of the two ankle pressures for each leg are used for the calculation. The ABI is calculated for both legs. A patient having an ABI of less than 0.9 is considered to have PAOD.

Functional impairments can be assessed using any standardized functional assessment tool (e.g., SF-36 or Walking Impairment Questionnaire).

### BRIEF DESCRIPTION OF THE DRAWINGS

FIG. 1 is a graph showing that mean Cₘₐₓ increased with rifalazil dose in males and females at doses ranging from 2.5 to 50 mg.

FIG. 2 is a graph showing that AUC from 0 to infinity (AUC_{0-∞}) increased with rifalazil dose in males and females at doses ranging from 2.5 to 50 mg.

FIG. 3 is a graph depicting rifalazil's Cₘₐₓ/dose, normalized to mg/kg.

FIG. 4 is a graph depicting rifalazil's AUC/dose, normalized to mg/kg.

### DETAILED DESCRIPTION OF THE INVENTION

Rifamycins

Rifamycins are compounds characterized by a chromophoric naphthohydroquinone group spanned by an aliphatic bridge. Exemplary rifamycins are rifalazil (3′-hydroxy-5′-(4-isobutyl-1-piperazinyl) benzoxazinorifamycin; also known as KRM-1648 or ABI-1648), rifampin, rifabutin, rifapentin, and rifaximin. Other rifamycins are disclosed in U.S. Pat. Nos. 4,690,919; 4,983,602; 5,786,349; 5,981,522; 6,316,433 and 4,859,661, U.S. Patent Application Nos. 60/341,130 and 60/341,591, and U.S. Patent Publication Nos. US2005-0043298 A1; US2005-0137189 A1; and US2005-0197333 A1, each of which is hereby incorporated by reference.

The structure of rifalazil is shown below.

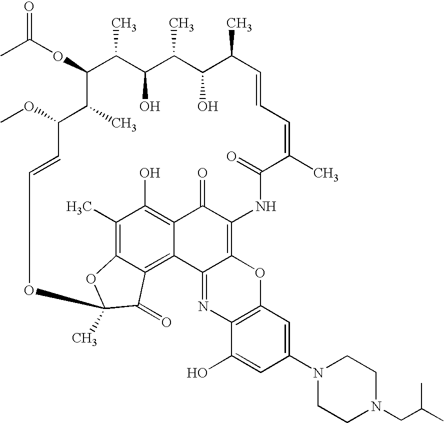

Rifalazil is a dark blue solid that is partially amorphous and partially crystalline. There is no observable melting point and no polymorphs have been detected.

Rifalazil is a highly lipophilic molecule having limited solubility in water at physiological pH (approximately 200 ng/mL). Evidence of the highly lipophilic behavior of the molecule is illustrated in the partition coefficient (n-octanol:water) of between 70, 569 and over 900,000 in different experiments (Log P range; 4.9-5.9).

Rifalazil degrades to a 25-desacetyl derivative under both acidic and basic conditions. Typical of ester hydrolysis, the degradation in highly alkaline solutions is rapid while at an acidic pH, e.g., pH 1, the degradation at room temperature is slower, approximately 6% in one hour.

The drug product currently being produced is a hard gelatin capsule containing rifalazil that has been formulated using microgranules made as described in U.S. Pat. No. 5,547,683. Materials Processing Technology Inc. (Patterson, N.J.) manufactures the granulated rifalazil which is subsequently encapsulated at ProClinical Pharmaceutical Services (Phoenixville, Pa.). This formulation for the 25 mg rifalazil capsules is summarized in Table 2, below.

TABLE 2

| | 25 mg | |
|---|---|---|
| | Strength | |
| | | Amount per |
| | | Dosage Unit |
| Ingredient | Function | (mg) |
| ABI-1648 Drug Substance | Active substance | 25.00 |
| Mannitol, USP | Binder & Filler | 106.93 |
| Colloidal Silicon Dioxide, NF | Glidant | 0.63 |
| Hydroxypropyl Cellulose, USP | Binder, granulating agent | 0.89 |
| Water, USP | Solvent | 0.65 |
| Polysorbate 80, NF | Surfactant, wetting agent | 0.16 |
| Magnesium Stearate, NF | Lubricant | 0.67 |
| Total Capsule Fill Weight | | 134.93 |

Use of Rifamycins in the Treatment of PAOD

The use of rifalazil in the treatment of PAOD is supported by both preclinical and clinical lines of evidence. First, in a rabbit model of atherosclerosis in which C. pneumoniae infection exacerbated plaque deposition, treatment with rifalazil resulted in reduced C. pneumoniae burden and plaque area stenosis compared with placebo-treated animals (see below). Second, the clinical efficacy of rifalazil in eradication of chlamydial infection has been successfully demonstrated in a Phase 2 study in men with non-gonococcal urethritis in which a single oral dose eradicated Chlamydia in >86% of patients. Third, rifalazil is 2000 times more potent against C. pneumoniae than roxithromycin, a potent anti-chlamydial agent currently registered in European countries, which has been shown to be effective in the treatment of patients with PAOD when administered for 28 days.

In addition, based on overall safety data in animals and man, we believe that a dose of rifalazil can safely be administered, tolerated, and will likely result in clinical benefit, measured by improvement in peak walking time, through its anti-Chlamydial action in these patients.

Animal Studies in Atherosclerosis

A study in rabbits demonstrated that rifalazil has effects on the acceleration of atherosclerosis induced by chlamydial infection in a rabbit model fed a high cholesterol diet.

Forty-five rabbits were fed a modestly enhanced (25%) cholesterol diet. Thirty received three separate C. pneumoniae inoculations performed at 3-week intervals. Similarly, 15 control rabbits were intranasally inoculated with 1 mL of normal saline under the same conditions. Three days after final inoculation, rabbits were assigned to treatment groups as shown in Table 3.

TABLE 3

| Inoculate | (n) | Treatment Group |
|---|---|---|
| C. pneumoniae | 10 | Azithromycin, 30 mg/kg PO daily for 1 week; |
| | | 30 mg/kg PO twice weekly for 6 weeks |
| C. pneumoniae | 10 | Rifalazil, 5 mg/kg PO daily for 1 week; |
| | | 5 mg/kg PO twice weekly for 6 weeks |
| C. pneumoniae | 10 | Placebo, 1 mL normal saline/kg PO daily for |
| | | 1 week; 1 mL/kg PO twice weekly for 6 weeks |
| Normal saline | 5 | Azithromycin, 30 mg/kg PO daily for 1 week; |
| | | 30 mg/kg PO twice weekly for 6 weeks |
| Normal saline | 5 | Rifalazil, 5 mg/kg PO daily for 1 week; |
| | | 5 mg/kg PO twice weekly for 6 weeks |
| Normal saline | 5 | Placebo, 1 mL normal saline/kg PO daily for |
| | | 1 week; 1 mL/kg PO twice weekly for 6 weeks |

Nineteen weeks after beginning antibiotic treatment, the rabbits were euthanized. After euthanasia, hearts were examined histologically and by immunofluorescence. Animal serum samples were also examined for seropositivity to the infecting organism.

In a similar study published previously, C. pneumoniae-infected rabbits accumulated significantly more plaque than uninfected rabbits, and azithromycin treatment had a significant effect in preventing Chlamydia-induced plaque formation (Muhlestein, 2000). In the current study, the time after infection and treatment with either azithromycin or rifalazil was increased by seven weeks, a more rigorous test for the durability of treatment. Consistent with previously published work, C. pneumoniae-infected rabbits accumulated more plaque than non-infected rabbits (p=0.08). Rifalazil treatment reduced the infectious burden of Chlamydia in the vasculature, (p<0.001) as did azithromycin (p=0.005). In addition, rifalazil (p=0.08), but not azithromycin (p=0.94), trended towards producing significantly reduced plaque area stenosis compared with placebo-treated animals (Table 4).

TABLE 4

| | | Plaque Area | P value versus infected |
|---|---|---|---|
| Treatment Group | (n) | Stenosis (%) | placebo |
| Infected, placebo-treated | 10 | 29.0 | |
| Uninfected animals | 10 | 22.8 | p = 0.08 |
| Infected, azithromycin- | 10 | 30.2 | p = 0.94 |
| treated | | | |
| Infected, rifalazil-treated | 10 | 24.2 | p = 0.13 |

To date, we have evaluated the pharmacokinetics (PK) of both single and multiple doses of rifalazil in three Phase 1 studies (Table 5). Study ABI-1648-006 was conducted to evaluate the PK of single and multiple oral doses of 2.5, 5, 12.5, and 25 mg administered to healthy male and female volunteers. Two of the Phase 1 studies were performed to evaluate specific PK considerations for rifalazil: Study ABI-1648-007 compared the bioavailability of a single 50-mg oral dose when administered to healthy male and female volunteers to ascertain any gender differences and Study ABI-1648-009 evaluated the effect of fasted and fed conditions on the PK of a single 25-mg oral dose in healthy male volunteers.

TABLE 5

| Study | Pharmacokinetic | Patient | Food | |
|---|---|---|---|---|
| Number | Objective | Population | Condition | Doses Evaluated |
| ABI-1648- | Evaluate single | Healthy male | Standard | Single Doses: 2.5, 5, |
| 006 | and multiple dose | and female | meal | 12.5, 25 mg |
| | PK parameters for | volunteers | | Multiple Doses: 2.5 QD × |
| | single and | | | 5 days, 5 QD × 5 days, |
| | multiple doses | | | 12.5 × 2 doses separated |
| | | | | by 72 hours |
| ABI-1648- | Compare | Healthy male | Standard | Single Dose: 50 mg |
| 007 | bioavailability and | and female | meal | |
| | PK between males | volunteers | | |
| | and females | | | |
| ABI-1648- | Compare | Healthy male | Fasted; | Single Dose: 25 mg |
| 009 | bioavailability and | volunteers | Standard | |
| | PK between | | meal; | |
| | different meal | | High-fat | |
| | conditions | | meal | |

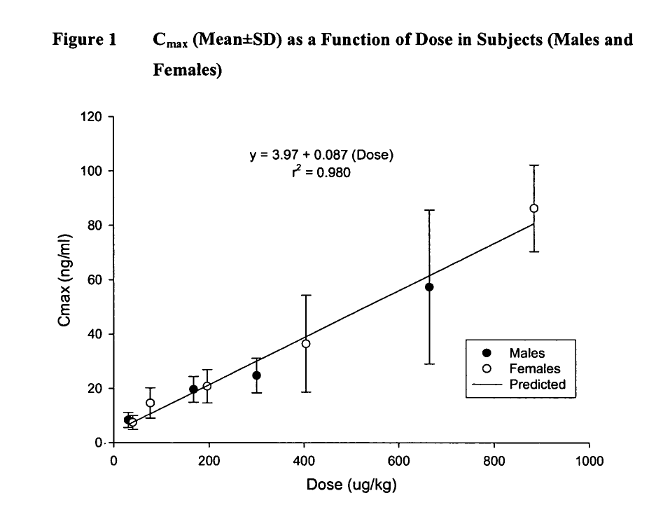

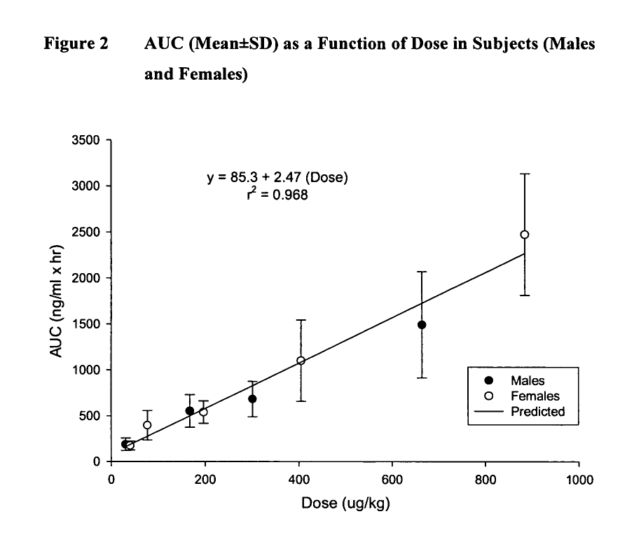

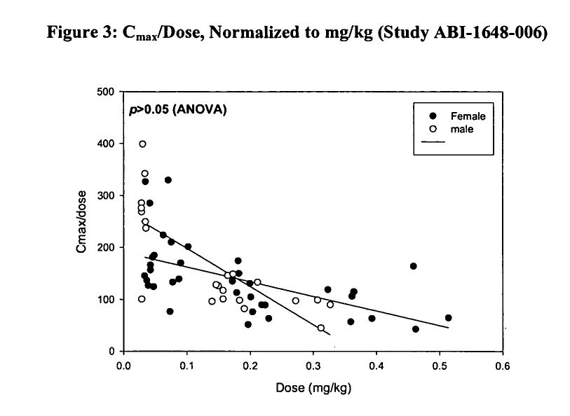

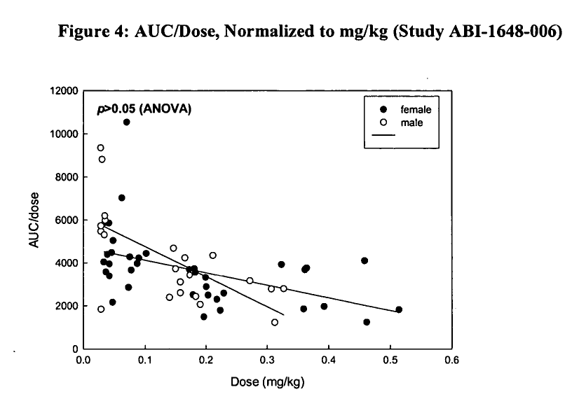

Exposure to rifalazil, as assessed by mean Cmax and AUC from 0 to infinity (AUC_{0-∞}), increased with rifalazil dose in both males and females at doses ranging from 2.5 to 50 mg (FIG. 1 and FIG. 2, respectively); however, the increase was not dose proportional, especially at the higher doses. There were no significant differences between males and females for AUC_{0-∞} when data were normalized to 1 mg/kg following single doses of 2.5 to 50 mg rifalazil (see FIG. 3 and FIG. 4). However, following multiple doses of 2.5, 5, and 12.5 mg, males exhibited significantly higher AUC from 0 to the dosing interval (AUC_{0-Tau}) (normalized to 1 mg/kg) as compared with females.

Pharmacokinetic analyses by gender show that after either single or multiple doses with rifalazil ranging from 2.5-50 mg, mean time to maximum concentration (Tₘₐₓ) was similar for males and females and ranged from approximately 4-6 hours. The Vd_{β}/F was large indicating extensive distribution in the body. The mean elimination t_{1/2} ranged from approximately 100-150 hours (i.e., approximately 4 to 6 days) following single doses. Following multiple dosing with up to 5 days of study drug, the mean elimination t_{1/2} ranged from approximately 87-174 hours. The mean minimum plasma concentrations (Cₘᵢₙ) of rifalazil over time during 5 days of dosing with 2.5 and 5 mg rifalazil increased from Days 2-6 in both males and females at both dose levels. Based on these data, it does not appear that steady state was reached after 5 days of dosing.

A food effect was observed when a single 25-mg rifalazil dose was administered following either a standard (30% fat) or high fat (60% fat) meal compared with administration of rifalazil following an overnight fast. Analyses of Cₘₐₓ and AUC under fed versus fasting conditions showed that absorption of rifalazil was lower when subjects were administered the study drug after an overnight fast as compared with following either a 30% or a 60% fat meal; in addition, Tₘₐₓ was shorter, occurring at approximately 3 hours.

Table 6 presents mean PK parameters obtained following single oral doses of rifalazil 2.5, 5, 12.5, and 25 mg administered to male and female subjects. As shown, mean Cₘₐₓ and AUC_{0-∞} increased with rifalazil dose in both males and females (FIG. 1 and FIG. 2); however, the increase was not dose proportional at the majority of doses tested (FIG. 3 and FIG. 4). Mean Tₘₐₓ ranged from 4.8-6.2 hours. The mean distribution t_{1/2} ranged from 4.5-8.6 hours and mean elimination t_{1/2} from approximately 101 -150 hours; these parameters did not differ between males and females. Mean clearance (Cl/F) and Vd_{β}/F increased as a function of dose, likely due to a decrease in fractional absorption (F), and were similar between males and females.

TABLE 6

| | | Males | | | Females | | | |
|---|---|---|---|---|---|---|---|---|
| | | | 12.5 | | | | 12.5 | |
| | | 2.5 mg | mg | 25 mg | 2.5 mg | 5 mg | mg | 25 mg |
| Parameter | Stat | (N = 8) | (N = 10) | (N = 8) | (N = 10) | (N = 8) | (N = 11) | (N = 8) |
| Cₘₐₓ | Mean | 8.4 | 19.7 | 24.7 | 7.5 | 14.6 | 20.8 | 36.5 |
| (ng/mL) | SD | 2.81 | 4.75 | 6.39 | 2.58 | 5.56 | 6.12 | 17.85 |
| | GMean | 7.8 | 19.2 | 23.9 | 7.1 | 13.5 | 19.9 | 33.3 |
| AUC_{0-∞} | Mean | 187.8 | 551.9 | 682.7 | 174.3 | 397.0 | 540.28 | 1101.5 |
| (ng/mL · hr) | SD | 68.70 | 177.81 | 193.17 | 48.45 | 160.47 | 122.31 | 442.63 |
| | GMean | 171.5 | 528.0 | 655.9 | 168.1 | 372.7 | 525.5 | 1024.6 |
| Tₘₐₓ | Mean | 5.3 | 5.0 | 5.0 | 4.8 | 5.0 | 6.2 | 4.8 |
| (hr) | SD | 1.49 | 1.05 | 1.85 | 1.03 | 1.07 | 2.27 | 1.04 |
| Distribution t_{1/2} | Mean | 5.3 | 7.7 | 8.7 | 5.0 | 4.5 | 6.1 | 7.8 |
| (hr) | SD | 1.58 | 0.39 | 2.08 | 2.02 | 1.98 | 1.28 | 1.69 |
| Elimination t_{1/2} | Mean | 101.1 | 133.6 | 116.4 | 133.1 | 136.8 | 110.3 | 150.5 |
| (hr) | SD | 39.46 | 34.90 | 34.30 | 69.04 | 59.24 | 24.32 | 56.31 |
| (Cl/F) | Mean | 0.21 | 0.33 | 0.48 | 0.25 | 0.23 | 0.39 | 0.43 |
| (L/hr/kg) | SD | 0.140 | 0.095 | 0.162 | 0.086 | 0.081 | 0.110 | 0.203 |
| Vd_{β}/F | Mean | 25.4 | 62.3 | 74.2 | 45.0 | 41.6 | 61.6 | 82.8 |
| (L/kg) | SD | 8.44 | 17.86 | 11.13 | 21.84 | 13.33 | 18.92 | 22.37 |
| Cₘₐₓ/Dose | Mean | 269.6 | 117.4 | 82.0 | 183.1 | 185.1 | 106.9 | 91.3 |
| | SD | 86.6 | 22.6 | 19.1 | 68.5 | 75.7 | 37.8 | 41.3 |
| AUC_{0-∞}/Dose | Mean | 6084.0 | 3299.8 | 2273.8 | 4274.6 | 5124.5 | 2759.4 | 2796.3 |
| | SD | 2307.2 | 927.2 | 633.7 | 1134.3 | 2493.1 | 755.7 | 1175.8 |

Gmean = Geometric mean

As shown in Table 6, FIG. 3, and FIG. 4, Cₘₐₓ/Dose and AUC_{0-∞}/Dose decreased as a function of dose suggesting decreased bioavailability as a function of increasing dose. There were no statistically significant differences between males and females for Cₘₐₓ/Dose and AUC_{0-∞}/Dose across doses (FIG. 3 and FIG. 4), based on the non-significant p-value for the gender by dose interaction from an analysis of variance (ANOVA) model using gender, dose (mg/kg), dose², and the interaction of gender with dose (gender by dose p-value=0.1962, 0.2068 for Cₘₐₓ/Dose and AUC_{0-∞}/Dose, respectively). There also were no statistically significant differences between males and females for analysis of elimination t_{1/2} or Cl/F for any dose. The Vd_{β}/F was significantly lower for males receiving 2.5 mg as compared with females; however, no differences were observed in Vdβ/F between males and females after dose adjustment based on 1 mg/kg for the 2.5 mg dose. In addition, no differences were observed in Vdp/F between males and females for the 12.5- or 25-mg doses.

Table 7 presents a summary of the PK parameters obtained following multiple oral doses of rifalazil 2.5 mg for 5 days, 5 mg for 5 days, and 12.5 mg for 2 doses (separated by 72 hours) in male and female subjects. Following multiple oral doses, mean Cₘₐₓ and AUC_{0-∞} increased with rifalazil dose in both males and females, however, the increase was neither linear nor proportional. Mean Tₘₐₓ ranged from 4.0-6.3 hours and did not appear to differ between males and females. The mean elimination t_{1/2} ranged from 118-134 hours in males and from 86-140 hours in females; the differences were not significant.

Statistical analysis of the multiple dose PK parameters was also conducted. There were no significant differences in Cₘₐₓ between males and females at dose levels of 2.5, 5.0, and 12.5 mg. Significant differences in AUC_{0-Tau} were observed between males and females for the 2.5-mg multiple dose group; however, AUC_{0-Tau} values were not significant for the 5.0- or 12.5-mg multiple dose groups.

TABLE 7

| | | Males | | | Females | | |
|---|---|---|---|---|---|---|---|
| | | 2.5 mg × 5 | 5 mg × 5 | 12.5 mg × 2 | 2.5 mg × 5 | 5 mg × 5 | 12.5 mg × 2 |
| Parameter | Stat | (N = 8) | (N = 8) | (N = 10) | (N = 7) | (N = 8) | (N = 6) |
| Cₘₐₓ last dose | Mean | 8.5 | 17.3 | 20.9 | 7.0 | 14.3 | 19.1 |
| (ng/mL) | SD | 1.56 | 4.81 | 7.63 | 2.47 | 7.15 | 4.88 |
| AUC_{0-Tau} | Mean | 178.5 | 260.7 | 398.4 | 98.2 | 196.9 | 339.0 |
| (multiple) | | | | | | | |
| (ng/mL · hr) | SD | 67.60 | 58.07 | 136.75 | 15.55 | 85.93 | 127.88 |
| Tₘₐₓ last dose | Mean | 4.3 | 4.0 | 6.2 | 4.3 | 5.3 | 6.3 |
| (hr) | SD | 1.28 | 1.51 | 2.57 | 2.14 | 1.49 | 2.94 |
| Elimination t_{1/2} | Mean | 134.4 | 118.6 | 120.3 | 140.2 | 173.6 | 86.5 |
| (hr) | SD | 100.8 | 56.2 | 48.0 | 44.0 | 137.2 | 15.7 |
| | Mean | 279.2 | 273.7 | 124.2 | 184.1 | 183.6 | 90.6 |
| Cₘₐₓ/Dose | SD | 64.5 | 66.4 | 38.4 | 79.3 | 99.7 | 24.9 |
| | Mean | 5788.5 | 4152.6 | 2357.9 | 2540.1 | 2539.0 | 1614.0 |
| AUC_{0-Tau}/Dose | SD | 2278.35 | 946.77 | 642.44 | 511.64 | 1278.35 | 626.87 |

AUC_{0-Tau} = AUC for 24 hours after the last dose for the 2.5 and 5 mg groups and 72 hours after the last dose for the 12.5 mg group.

Table 7 also presents results of the comparison between males and females of Cₘₐₓ and AUC_{0-Tau} normalized to 1 mg/kg following multiple doses of 2.5, 5.0, and 12.5 mg. Both Cₘₐₓ and AUC_{0-Tau} were significantly higher for males compared with females for the 2.5- and 5-mg doses. As well, AUC_{0-Tau} was significantly higher for males as compared with females for comparison of the 12.5-mg dose.

The mean Cₘᵢₙ of rifalazil over time during 5 days of dosing with 2.5 and 5 mg rifalazil increased from Days 2-6 in both males and females at both the 2.5- and 5-mg dose levels. Based on these data, steady state was not reached after 5 days of dosing.

Based on the results of this study it was concluded that:
Mean Cₘₐₓ and AUC following both single and multiple oral doses of 2.5, 5.0, 12.5, and 25.0 mg increased with dose; however, the increase was not dose-proportional.
Time to maximum concentration ranged from approximately 4-6 hours after single dose administration.
The Vd_{β}/F was large indicating wide tissue distribution and the elimination t_{1/2} ranged from approximately 100-150 hours (i.e., approximately 4-6 days).
There were no significant differences in AUC and Cₘₐₓ between males and females administered single doses, after data were normalized for body weight; Tₘₐₓ and elimination t_{1/2} were also not different across gender. However, following multiple doses of 2.5, 5.0, and 12.5 mg, a significantly higher AUC (normalized for body weight) was observed in males compared with females.
The mean Cₘᵢₙ of rifalazil over time during 5 days of dosing with 2.5 and 5.0 mg rifalazil increased from Days 2-6 in both males and females at both the 2.5- and 5.0-mg dose levels; therefore, steady state was not reached after 5 days of dosing.

Oral administration of 25 mg of rifalazil demonstrated significant anti-chlamydial activity in patients with NGU and was well tolerated. Due to the recalcitrant response of C. pneumoniae to antibiotic therapy, the duration of therapy is thought to be an important parameter for optimizing treatment. Previous large well-controlled randomized studies have tested long term antibiotic therapy in the CHD population. A longer term treatment schedule with rifalazil than was utilized in eradicating C. trachomatis in NGU (single dose) may represent a preferred therapeutic regimen for producing a clinically beneficial effect on C. pneumoniae in patients with PAOD. Further, dosing of PAOD patients with rifalazil for a duration of 8 weeks may further optimize therapeutic effect against C. pneumoniae in these patients. Chlamydia pneumoniae infects the vasculature, and to elicit significant antibacterial activity against this pathogen, longer courses of treatment than those directed at C. trachomatis (i.e., single dose) are preferred. Pharmacokinetic and pharmacodynamic modeling predict that this dosage will provide continuous plasma and tissue concentrations of rifalazil above the MIC₉₀ for C. pneumoniae for over 9 weeks. A single dose of 25 mg rifalazil administered once per week for up to 4 weeks was not associated with significant adverse effects in Phase 1 studies. Single-dose data on the 25-mg dose from a Phase 2 clinical study also supports the safety and efficacy of this dose level as an anti-chlamydial therapy. Furthermore, Phase I studies in volunteers showed that the overall incidence of adverse effects with rifalazil 25 mg dosing decreased with subsequent doses as compared with the initial dose. This incidence achieved a zero percent incidence of side effects in volunteers with the fourth weekly dose of once a week dosing. Consequently, the optimal dose in patients with PAOD is considered to be 25 mg administered as a single dose, once a week, for a total of 8 weeks. Collectively, clinical safety data for rifalazil, the large margin between preclinical NOAEL doses in animals and clinical doses, and the significant anti-chlamydial activity of rifalazil make this a particularly useful therapeutic treatment regimen for the treatment of patients with PAOD who are seropositive for C. pneumoniae.

Non-Clinical Pharmacokinetics

Since the PK profile of rifalazil has been studied in man, only a brief summary of the non-clinical PK is provided. Overall, the absorption, distribution, metabolism, and elimination of rifalazil observed in the animals studied are similar to those observed in man.

A series of studies also was performed to evaluate the in vitro metabolism of rifalazil by human microsomes as well as to determine the inhibition and/or induction potential of rifalazil on human cytochrome P450. In humans, rifalazil metabolizes to the M1 metabolite and to the M4 metabolite, both of which are active and inhibit the growth of Mycobacterium tuberculosis and Mycobacterium avium complex. The overall extent of rifalazil metabolism is very low, accounting for less than 3% of the administered dose.

After oral administration, both Cₘₐₓ and AUC in all species evaluated were dose-dependent but not dose-proportional suggesting that absorption of rifalazil from the GI tract may have been saturable or limited due to the lack of solubility with rifalazil. Peak plasma concentrations were observed from 4 to 18 hours after oral administration.

The oral bioavailability of rifalazil in the rat was dose-dependent and ranged from 6.1% to 11.7% at doses of 100 mg/kg and 28.9% and 43.5% at doses of 3 mg/kg in female and male rats, respectively. The oral bioavailability of rifalazil in the beagle dog following doses of 10 mg/kg was approximately 16% to 17%.

Extensive tissue distribution was observed in rats following a single oral dose. Distribution to the majority of tissues was higher than observed plasma levels. Following a single oral dose of rifalazil administered to rats (3 mg/kg), the highest tissue concentrations were observed in the wall of the GI tract (5.4 μg/gm) at 1 hour and in the spleen (5.1-9.3 μg/gm) at 8-12 hours post dose (Study 6510-109).

In rats and dogs, the Vd_{β}/F following oral administration was 40-80 L/kg. Tissue exposure, expressed as AUC, ranged from a tissue to plasma ratio of 4.9-33.7 (in testes and liver, respectively). The highest mean Cₘₐₓ observed was in the wall of the GI tract and occurred approximately 1 hour post-dose. Concentrations of rifalazil in genitourinary and GI tissues as well as in coronary arteries after multiple oral doses in the monkey exceeded concentrations in plasma. These data suggest that, in man, rifalazil is likely to be distributed into a large tissue compartment and the concentrations would exceed those in plasma. A large tissue compartment of rifalazil is considered important for efficacy against Chlamydia given the intracellular nature of Chlamydia infection.

Overall, in rats, mice and dogs, rifalazil was distributed throughout the body and concentrations were observed in plasma and tissue at levels that may be effective in the treatment of the targeted bacteria (i.e., Chlamydia spp., H. pylori, and C. difficile).

Studies Using Single Doses of 25 mg

Rifalazil has been shown to be well tolerated in both healthy volunteers and patients when administered both in single-dose studies of 25 mg, and in multiple-dosing regimens using weekly administration of a single 25-mg dose. Other clinical studies where cumulative doses of 25 mg have been administered using different daily dosing regimens also support the tolerability of a weekly 25-mg dose level. These studies are described briefly below.

Several Phase 1 studies of rifalazil administration have been carried out using 25 mg single doses. In one such study, sixteen patients received single doses of 25 mg. Overall, 75% of patients (12/16) reported treatment-emergent adverse events (AEs). The overall incidence of AEs did not differ between dose levels in this study (2.5, 5, 12.5, or 25 mg), nor compared to the placebo group (15/21, 71%). However, for all dose groups, the incidence of AEs was higher for females than for males, and this difference was also observed in the placebo group. At 25 mg, 88% of females (31/39) and 55% of males (16/29) reported treatment-emergent AEs. The AEs with an incidence greater than or equal to 10% were:

Headache (38%)

Lymphocyte count decreased (31%)

Body temperature increased (25%)

Bacteria in urine (19%)

White blood cells (WBCs) in urine (19%)

Nausea (13%)

Pharyngitis (13%)

Neutrophil count increased (13%)

Blood present in urine (13%)

Monocyte count increased (13%)

In another study, a single oral dose of 25 mg rifalazil was administered and its effects on ethinyl estradiol and norethindrone levels administered as the contraceptive Ortho-Novum 1/35® in 16 healthy post-menopausal female volunteers for 14 days was determined. The incidence of AEs was similar between the two treatment periods, with 13 (93%) of the 14 subjects included in the safety population experiencing at least 1 AE during dosing with Ortho-Novum 1/35 alone and 12 (86%) during dosing with rifalazil and Ortho-Novum 1/35. Overall, 4 (29%) subjects experienced at least 1 AE considered by the Investigator to be at least possibly related to rifalazil. The most commonly reported rifalazil-related events were nausea (4 subjects, 29%); headache (3 subjects, 21%); and loose stools, feeling hot, rigors, and myalgia (2 subjects each, 14%).

The majority of AEs were of mild or moderate intensity. One subject experienced myalgia of severe intensity during dosing with rifalazil and Ortho-Novum 1/35 that was assessed as probably related to study medication. No deaths or other serious AEs were reported during the study. Two subjects withdrew from the study because of AEs during the Ortho-Novum 1/35 only dosing period; neither subject received rifalazil.

No clinically meaningful changes were noted for mean hematology or clinical chemistry parameters during the study. Transient Grade 3 decreases in absolute lymphocyte count (ALC<0.5×10³/mm³) were observed in two subjects, both of which occurred the day following the rifalazil dose; no Grade 3 or 4 neutropenia or leukopenia was observed during the study.

In another study, a single 25-mg oral dose of rifalazil was administered to healthy male adults under one of three distinct food conditions: a high fat meal (60% fat), standard meal (30% fat), and under fasting conditions. No placebo-control was used in this study. Twelve subjects were enrolled, 10 of whom received all doses of rifalazil. Although high fat meal conditions result in higher plasma levels of rifalazil, the incidence of AEs was similar across all food conditions. A total of 83% of subjects (10/12) experienced treatment-emergent AEs, all of mild to moderate intensity. Fifty-eight percent were considered by the investigator to be drug-related. The most common reported drug-related AEs were back pain (25%) and headache (17%), and these were reported only in subjects under fed conditions. All other study medication-related AEs were reported by one subject (8%) only.

In a placebo-controlled study of 30 mg and 100 mg in 6 healthy volunteers, five patients received the 30 mg dose of rifalazil. Sixty-two percent of patients (5/8) reported AEs while 33% of placebo subjects (3/9) reported AEs. The only AE of note reported in this rifalazil dose group was headache (38%, 3/5 subjects) compared to the placebo-treated group (11%, 1/9 subjects). In the group treated with a 30-mg single oral dose of rifalazil, one subject had a baseline WBC count of 4.3×10³ cells/mm³ that was just below the lower limit of normal (4.5×10³ cells/mm³). However, this baseline value was not considered clinically significant nor were subsequent nadir values obtained on Days 3 and 7 (3.8×10³ cells/mm³).

Studies Using Multiple Weekly Doses of 25 mg Rifalazil

In one study, healthy volunteers received 25 or 50 mg once a week for 4 weeks. The overall incidence of AEs was 33% after the first weekly dose, 12% after the second and third weekly dose, and 0% at the fourth weekly dose. No subject discontinued the study due to AEs, and no serious AEs were reported in this study. The predominant AEs reported in the rifalazil group but not in the placebo group included: back pain (2/6. 33%), chills (3/6, 50%), neck pain (1/6. 17%), insomnia, and a range of gastrointestinal (GI) disturbances such as anorexia, nausea, thirst and vomiting (all in 1/6 patients, 17%). Rifalazil-treated groups had a higher number of patients than placebo report the following: asthenia (33% vs 25%), fever (67% vs 25%), headache (67% vs 50%), pain (50% vs 25%), and dizziness (50% vs 25%).

Low WBC counts were noted for three of six subjects treated with 25 mg/week and 6 of 8 subjects treated with 50 mg/week in this study. The lowest individual value for subjects receiving 25 mg/week was 2.2×10³/mm³ (Day 25), and for 50-mg/week subjects, 2.3×10³/mm³ (Day 11). All values returned to baseline within 2 weeks of cessation of dosing. A transient increase in mean values for aspartase aminotransferase (AST) and alanine aminotransferase (ALT) was observed in the 50 mg/week group, but not in the 25 mg/week dose group. The above normal values at 50 mg/week occurred in 1 subject, and were of moderate Grade 2 severity during the interval from approximately Day 15 to Day 78. Grade 4 lymphocyte toxicity was observed in 1 subject in the 25-mg/week dose group and in 3 subjects in the 50-mg/week dose group. All values returned to baseline levels within 2 weeks of cessation of dosing.

The phenomenon of accommodation or tolerance with repeat weekly dosing of rifalazil became evident during the course of the study for both the 25-mg and 50-mg doses. In the 25-mg dosage group, the peak incidence of AEs occurred in the first week and decreased substantially over the remaining weeks. Rifalazil 25 mg/week was judged to be fairly well tolerated with accommodation occurring during the second week. The dose regimen of 50 mg/week was not considered well tolerated, although tolerance to this dosing regimen was also noted.

Studies with 25 mg Cumulative Doses Over One Week

Different dosing regimens of rifalazil that achieve 25-mg cumulative doses in 1 week also provide relevant clinical information on the safety of 25 mg administered as a single dose once per week.

For a cumulative dose of 25 mg, the overall incidence of AEs was somewhat lower when rifalazil was administered as 5 mg QD for 5 days, compared with a single dose of 25 mg (75%). Similarly, the overall incidence of drug-related AEs was lower when 25 mg was administered as a 5 mg for 5 days (63%), vs. 12.5 mg as 2 doses (separated by 72 hours) (70%), and as a single dose of 25 mg (75%). However, in terms of types of AEs, the 5 mg QD for 5 days regimen had a greater incidence of flu-like symptoms (5/15 patients, 33%), than the 12.5 mg×2 dose regimen (15%), and the 25 mg single-dose administration (3/16 patients, 19%).

Studies Using 25 mg Rifalazil

In another study, three dose levels of rifalazil were administered for the treatment of non-gonococcal urethritis in 170 male patients. Doses used were in this study were single-doses of 2.5, 12.5 mg and 25 mg. Males were between 18-45 years of age with signs or symptoms of urethritis, including urethral discharge. Of the 128 patients in the rifalazil groups, 43 patients received 2.5 mg, 42 patients received 12.5 mg, and 43 patients received 25 mg. No deaths, serious AEs, or discontinuations due to AEs were reported in this study. The majority of AEs were rated as mild or moderate intensity. Four patients treated with 25 mg rifalazil and one patient treated with azithromycin experienced a severe AE. The most frequent side effect encountered with this dose was headache, which occurred in a treatment-emergent manner in 14% of patients.

In the 25-mg rifalazil group, the severe AEs included headache in two patients, dizziness, scarring, and neutropenia of grade 3 intensity in three patients (baseline less than the lower limit of normal for all three). The incidence of AEs considered possibly related to study drug was similar between the total rifalazil group (23%) and the azithromycin group (24%). Among rifalazil-treated patients, the incidence of drug-related AEs was 14%, 21%, and 35% in the 2.5 mg, 12.5 mg, and 25 mg rifalazil groups, respectively. No clinically significant differences were noted among rifalazil dose groups or between rifalazil groups and the azithromycin group for changes from baseline in any hematology or clinical chemistry parameters. The incidence of clinically significant laboratory abnormalities was similar between rifalazil-treated patients (21%) and azithromycin-treated patients (21%). Three patients, all in the 25 mg rifalazil group with below normal absolute neutrophil counts (ANCs) at enrollment, had decreases to Grade 3 neutropenia; none of these decreases were associated with clinical effects.

Pharmacokinetic Data to Support 25 mg Dose of Rifalazil

Rifalazil has a relatively long t_{1/2} of approximately 100 hours. Different doses studied have shown some differences in the terminal elimination t_{1/2}, with daily dosing associated with a slightly shorter t_{1/2}. Antibiotics with similar long t_{1/2}s and extensive tissue distribution are dosed on a weekly or biweekly basis for efficacy. Dalbavancin has a distribution and elimination t_{1/2} of approximately 180 hours and is dosed once a week based on data that showed serum bactericidal activity to persist at 7 days after dosing against target pathogens. Pharmacokinetic models of rifalazil dosed at 25 mg on a weekly basis show maintenance of serum bactericidal concentrations well above the MIC₉₀ for C. pneumoniae at 7 days as well.

We recently completed a PK simulation study for rifalazil, 25 mg, once weekly for a period of eight weeks. The PK simulation suggests that the rifalazil trough level increases from 1.3 to 2.6 ng/mL gradually after 5 weekly doses. Rifalazil concentrations would reach the steady-state (theoretically 96.8%) after the fifth dose, with trough levels remaining at approximately 2.6 ng/mL (from 2.6 to 2.67 ng/mL, 97.4%). The results of this simulation analysis indicate that the plasma concentration of rifalazil should remain well above the MIC₉₀ for C. pneumoniae throughout the eight week dose regimen period for the treatment of PAOD with rifalazil.

Pharmacodynamic Data to Support a 25 mg Dose of Rifalazil

Rifalazil appears to have a significant post-antibiotic effect and mechanistically demonstrates both concentration and time-dependent bactericidal mode of action. Single doses of rifalazil in cell culture studies demonstrate a protective effect against re-infection with C. trachomatis for up to 12 days, unlike macrolide comparators, among them azithromycin. The t_{1/2} of azithromycin is 65 hours, and it was dosed once weekly in the large, but negative, “Azithromycin for the Secondary Prevention of Coronary Events” (ACES) trial.

### Other Embodiments

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What is claimed is:

1. A method for treating peripheral arterial occlusive disease (PAOD) in a patient in need thereof by administering to said patient a rifamycin in an amount effective to treat PAOD in said patient.

2. A method for increasing the peak walking time (PWT) in a patient in need thereof by administering to said patient a rifamycin in an amount effective to increases the PWT.

3. A method for increasing the painless walking distance (PWD) in a patient in need thereof by administering to said patient a rifamycin in an amount effective to increases the PWD.

4. A method for reducing the occurrence and/or severity of intermittent claudication in a patient in need thereof by administering to said patient a rifamycin in an amount effective to reduce the occurrence and severity of intermittent claudication.

5. A method for reducing the functional impairments associated with the progression of PAOD in a patient by administering to said patient a rifamycin in an amount effective to reduce the functional impairments accompanying the progression of PAOD.

6. A method for reducing the number and/or frequency of vascular interventions over time and related clinical complications over time in a patient having PAOD as compared with an untreated age-, risk-, and diseased-matched patient, by administering to said patient a rifamycin in an amount effective to reduce the number and/or frequency of vascular interventions over time and related clinical complications over time in said patient.

7. A method for reducing the number and/or frequency of cardiovascular complications over time in a patient having PAOD as compared with an untreated age-, risk-, and diseased-matched patient, by administering to said patient a rifamycin in an amount effective to reduce the number and/or frequency of cardiovascular complications over time in said patient.

8. A method for reducing localized inflammation in an atherosclerotic plaque in a patient having PAOD by administering to said patient a rifamycin in an amount effective to reduce localized inflammation in a plaque.

9. A method for reducing the size of an atherosclerotic plaque in a patient having PAOD by administering to said patient a rifamycin in an amount effective to reduce the size of an atherosclerotic plaque.

10. A method of reducing the level of an inflammatory biomarker in a patient having PAOD by administering to said patient a rifamycin in an amount effective to reduce the level of said inflammatory biomarker.

11. A method of reducing the clinical complications associated with angioplasty and/or stent placement in a patient having PAOD by administering to said patient an effective amount of a rifamycin.

12. A method of reducing intimal hyperplasia and in-stent and peri-stent restenosis that occur after stent placement in a patient having PAOD by administering to said patient an effective amount of a rifamycin.

13. A method of reducing vascular smooth muscle cell proliferation and/or the cellular and molecular products of vascular smooth muscle cell proliferation in a patient having PAOD by administering to said patient an effective amount of a rifamycin.

14. A method of restoring endothelial function and capability in a patient having PAOD by administering to said patient an effective amount of a rifamycin.

15. The method of claim 14, wherein said patient has been diagnosed as having PAOD.

16. The method of claim 1, wherein said patient has not been diagnosed as having a bacterial infection that can be treated by administration of a rifamycin.

17. The method of claim 1, wherein said patient has been diagnosed as not having a bacterial infection that can be treated by administration of a rifamycin.

18. The method of claim 1, wherein said patient has been diagnosed as having an infection of C. pneumoniae.

19. The method of claim 1, wherein said patient is seropositive for Chlamydia pneumoniae.

20. The method of claim 1, wherein said rifamycin is rifalazil.

21. The method of claim 20, wherein said rifalazil is administered to said patient in an amount of between 12.5 and 50 mg, at a frequency of once per week for 4-10 weeks.

22. The method of claim 21, wherein said rifalazil is administered to said patient in an amount of 12.5-25 mg, at a frequency of once per week for 4-10 weeks.

23. The method of claim 21, wherein said rifalazil is administered to said patient in an amount of 12.5-25 mg, at a frequency of once per week for 8 weeks.

24. The method of claim 20, wherein said rifalazil is administered to said patient in an amount of between 12.5 and 50 mg, at a frequency of once per week for 4-10 weeks.

25. The method of claim 24, further comprising repeating said method every three to twelve months for at least six months and up to the lifetime of said patient.

26. The method of claim 20, wherein said rifalazil is administered at an initial dose of 2.5 to 100 mg once a week, for a period of two to 16 weeks, followed by a dose of 2.5 to 50 mg once a week, once each two weeks, once a month, or once each two months, for a period of at least six months and up to the lifetime of said patient.

27. The method of claim 1 further comprising administering to said patient one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), antipyretics (e.g., acetaminophen), or lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin).

28. The method of claim 27, wherein said additional agents are administered within 14 days, 7 days, 1 day, 12 hours, or 1 hour of administration of a rifamycin, or simultaneously therewith.
